(19) United States
(12) Patent Application Publication
(10) Pub. No.: US 2010/0094174 A1
(43) Pub. Date: Apr. 15, 2010

(54) METHOD FOR THREE-DIMENSIONAL BIOMECHANICAL DATA AND PARAMETER ANALYSIS AND SYSTEM USING THE SAME METHOD

(76) Inventors: Yong Jae Choi, Gangwon-do (KR); Im Sun Yang, Seoul (KR); Hong Seong Park, Seoul (KR); Donghee Choi, Gangwon-do (KR); Jar Young Choi, Gangwon-do (KR)

Correspondence Address: HAMMER & ASSOCIATES, P.C., 3125 SPRINGBANK LANE, SUITE G, CHARLOTTE, NC, 28226, US

(21) Appl. No.: 12/530,685

(22) PCT Filed: Mar. 13, 2008
(86) PCT No.: PCT/KR08/01409

§ 371 Date: Sep. 10, 2009

(30) Foreign Application Priority Data
Mar. 13, 2007 (KR) 10-2007-0024462
Aug. 14, 2007 (KR) 10-2007-0081888
Feb. 28, 2008 (KR) 10-2008-0018461

(51) Int. Cl. A61B 5/103

(52) U.S. Cl. 600/587

(57) ABSTRACT

A 3D biochemical data and parameter analysis method and system are provided. The 3D biochemical data and parameter analysis method, including: collecting first biomechanical information of an upper body and second biomechanical information of a lower body with respect to a standard movement; and providing 3D static data or 3D motion tracking data for a diagnosis or prognosis of a musculoskeletal system disease considering both the upper body and lower body, the 3D static data or 3D motion tracking data being generated by associating the collected first biomechanical information with second biomechanical information.

### TECHNICAL FIELD

The present invention relates to a biochemical analysis system, and more particularly, to a three dimensional (3D) biochemical data and parameter analysis method and system which comprehensively analyzes biochemical data, and thereby may accurately diagnose a musculoskeletal system disease and predict a disease onset.

### BACKGROUND ART

In general, an X-ray, magnetic resonance imaging (MRI), computed tomography (CT), and the like are used to diagnose a musculoskeletal system disease related to a foot or spine, or predict the disease possibility. However, such devices only provide measured result when a measurement target does not move, and may not measure biochemical information when a measurement target moves. Accordingly, diagnosis data may not be obtained while a measurement target is moving.

Biochemical analysis takes significant time and requires specialized equipment. Accordingly, biochemical analysis may not be used as diagnosis data for musculoskeletal system disease and disease onset.

For example, in a walking analysis where a biochemical analysis is applied, measurement for each specific body part such as legs, foot pressure, spine, and the like is available. A videotape analysis, exercise analysis based on a vision system using reflective markers, foot pressure analysis, and the like are used for a walk analysis.

For example, in a vision-based motion analysis system, general purpose products which may be used for animation, special effect of film, and the like are common. However, a vision-based motion analysis system may not generate biochemical data. Also, a data conversion process to a biochemical parameter is required to clinically apply raw visual data or raw technical data obtained using the vision-based motion analysis system. However, the above-described disadvantages are not overcome.

Analysis data in a biochemical analysis based on a doctor's diagnosis is not standardized. That is, a biochemical analysis depends on a personal proficiency of an expert. Accordingly, a reproducibility and objectivity of biochemical data obtained using a diagnosis device and method in a conventional art are inferior. Also, such disadvantages may not be overcome in a conventional art. Although devices to generate biochemical data have been developed and commercialized, the devices have other disadvantages. Specifically, the devices may measure and output only a portion of biochemical data, and may not simultaneously output dynamic and static data. Also, the devices may not output analysis data associated with biochemical data of other parts in a musculoskeletal system. That is, a system for outputting only a biochemical parameter required for treatment of musculoskeletal system has not been provided. An automatic prescription method based on a biochemical parameter is not expensive in comparison with a diagnosis device in a conventional art. Also, a system using the automatic prescription method such as an automatic recommended prescription system based on a biochemical parameter has not been developed. Moreover, a method to confirm whether a prescription is made according to a prescription of an expert has not been provided.

Thus, an automatic system which associates and analyzes biochemical data, interprets and outputs a biochemical parameter, prescribes and verifies a prescription with respect to an assistant device, and thereby may provide a more accurate prescription and a standard follow-up examination is required to diagnose and prescribe a musculoskeletal system disease and predict disease onset more accurately.

### DISCLOSURE OF INVENTION

### Technical Goals

The present invention provides a three dimensional (3D) biochemical data and parameter analysis method and system which simultaneously provides biochemical data of an upper body and lower body with respect to a diagnosis and prognosis of a biochemical musculoskeletal system disease, calculates and provides a biochemical parameter and automatic recommended treatment prescription, and confirms whether an expert's prescription is correctly made.

### Technical Solutions

According to an aspect of the present invention, there is provided a three dimensional (3D) biomechanical data and parameter analysis system, including: a biomechanical data and parameter analysis unit which collects first biomechanical information measured in an upper body and second biomechanical information measured in a lower body with respect to a standard movement, and provides 3D static data or 3D motion tracking data for a diagnosis or prognosis of a musculoskeletal system disease considering both the upper body and lower body, the 3D static data or 3D motion tracking data being generated by associating the first biomechanical information with second biomechanical information.

According to another aspect of the present invention, there is provided a 3D biochemical data and parameter analysis method, including: collecting first biomechanical information of an upper body and second biomechanical information of a lower body with respect to a standard movement; and providing 3D static data or 3D motion tracking data for a diagnosis or prognosis of a musculoskeletal system disease considering both the upper body and lower body, the 3D static data or 3D motion tracking data being generated by associating the collected first biomechanical information with second biomechanical information.

According to another aspect of the present invention, the 3D biochemical data and parameter analysis method of claim 16, further including: converting the first biomechanical information and second biomechanical information into a biochemical parameter to be clinically applied, and providing the diagnosis of the musculoskeletal system disease and a recommended prescription with respect to the diagnosed disease based on the biochemical parameter.

According to the present invention, various biochemical information is synchronized considering both an upper body and lower body for a diagnosis and prognosis of a biochemical musculoskeletal system disease, 3D static data or 3D motion tracking data is calculated, biochemical parameters are calculated, it is confirmed whether a prescription is made as expected, and thus more accurate diagnosis and prescription may be provided.

### Advantageous Effects

According to an embodiment of the present invention, a three dimensional (3D) biomechanical data and parameter analysis system and method three-dimensionally models and provides biochemical information considering both upper body biochemical information and lower body biochemical information, and thereby may diagnose and prognose a biochemical musculoskeletal system disease more accurately.

Also, according to an embodiment of the present invention, a 3D biomechanical data and parameter analysis system and method may perform a comprehensive diagnosis with respect to various diseases and symptoms in comparison with a musculoskeletal system disease diagnosis device in a conventional art, and efficiently and inexpensively calculate a biochemical parameter in comparison with a general purpose motion capture device.

Also, according to an embodiment of the present invention, a 3D biomechanical data and parameter analysis system and method provides biochemical information by associating upper body biochemical information with lower body biochemical information, provides basic data for a diagnosis, and thereby may embody a standard prescription and be widely used in various fields through a use of standard data.

### BRIEF DESCRIPTION OF DRAWINGS

FIG. 1 is a block diagram illustrating a configuration of a three dimensional (3D) biochemical analysis system according to an embodiment of the present invention;

FIG. 2 is a diagram illustrating an example of an attachment location of each marker to collect biochemical information;

FIG. 3 is a diagram illustrating a placement of the 3D biochemical analysis system of FIG. 1;

FIG. 4 is a flowchart illustrating a 3D biochemical analysis method according to an embodiment of the present invention;

FIG. 5 is a flowchart illustrating a method of collecting biochemical data in a vision system using a marker according to an embodiment of the present invention;

FIG. 6 is a diagram illustrating an example of an interface screen to provide an attachment location of each marker and biochemical parameter list;

FIG. 7 is a diagram illustrating an example of an interface screen to provide an attachment location of each marker to be attached to an anterior, posterior, right, and left side of a measurement target;

FIG. 8 is a diagram illustrating an example of an interface screen to provide an error state or omission state of a marker while verifying a location of each marker attached to a measurement target;

FIGS. 9 through 12 are diagrams illustrating a method of measuring upper body biochemical information;

FIGS. 13 and 14 are diagrams illustrating a method of measuring lower body biochemical information;

FIG. 15 is a diagram illustrating a measurement point of pelvis-related biochemical data;

FIG. 16 is a diagram illustrating a method of calculating a pelvis rotation angle;

FIG. 17 is a diagram illustrating a method of calculating a pelvis obliquity angle;

FIG. 18 is a diagram illustrating a method of calculating a pelvis tilting angle;

FIG. 19 is a diagram illustrating a method of calculating a pelvis elevation; and

FIGS. 20 and 21 are diagrams illustrating a method of determining a foot type.

### BEST MODE FOR CARRYING OUT THE INVENTION

Hereinafter, a three dimensional (3D) biomechanical data and parameter analysis system and method is described in detail by referring to the figures.

According to an embodiment of the present invention, upper body biochemical information and lower body biochemical information are simultaneously obtained for a diagnosis and prognosis of a biochemical musculoskeletal system disease, and comprehensive data is provided by associating the upper body biochemical information with lower body biochemical information.

The upper body biochemical information is at least one of a degree of finger bending, degree of arm bone curvature, shoulder elevation, degree of shoulder rotation, degree of shoulder tilting, curvature direction of a spine, degree of curvature of the spine, and difference in distance between an ear and shoulder.

Also, the upper body biochemical information is at least one of a pelvis rotation angle, pelvis obliquity angle, pelvis tilting angle, pelvis elevation, degree of bending of lower limbs, angle between a femur and a fibula, angle between a foot sole and vertex of calcaneal, angle between anklebone and knee joint, degree of toe bending, and foot pressure distribution.

To obtain the upper body biochemical information and lower body biochemical information, a standard movement such as standing up, walking, running, and the like is required of a measurement target, and the biochemical information may be calculated using data measured when the measurement target performs the standard movement.

According to an embodiment of the present invention, the upper body biochemical information and lower body biochemical information are comprehensively obtained for more accurate diagnosis and prognosis of the biochemical musculoskeletal system disease. Also, the comprehensive biochemical information is provided as 3D static data or 3D motion tracking data. Accordingly, a biochemical parameter is obtained.

First, a configuration of 3D biochemical data and parameter analysis system is described.

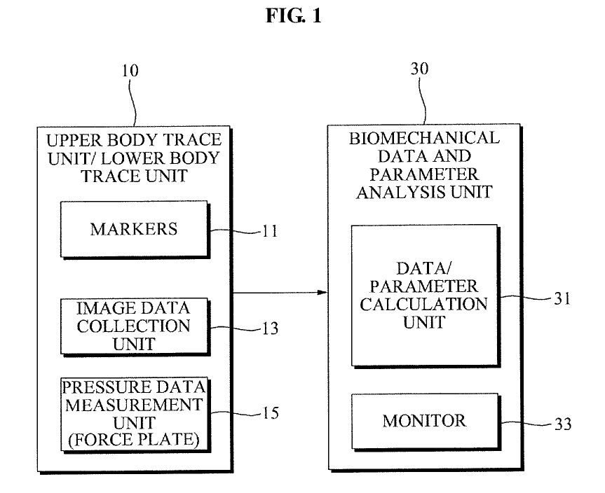

FIG. 1 is a block diagram illustrating a configuration of a 3D biochemical analysis system according to an embodiment of the present invention.

As illustrated, the 3D biochemical analysis system includes an upper body trace unit/lower body trace unit 10 and a biomechanical data and parameter analysis unit 30. The upper body trace unit/lower body trace unit 10 collects biochemical information with respect to a standard movement. The biomechanical data and parameter analysis unit 30 synchronizes the upper body biochemical information and lower body biochemical information collected from the upper body trace unit/lower body trace unit 10.

The upper body trace unit 10 includes a plurality of markers 11 and image data collection unit 13. The plurality of markers 11 is attached to an upper body of a measurement target. The image data collection unit 13 traces a location of each of the plurality of markers 11 according to the standard movement.

The plurality of markers 11 corresponds to any one of reflective markers or color markers. Also, the plurality of markers 11 is attached to a location of a skeleton in any one of hands, arms, shoulders, and spine considering desired biochemical information. The color markers may include black-and-white markers and disposable markers.

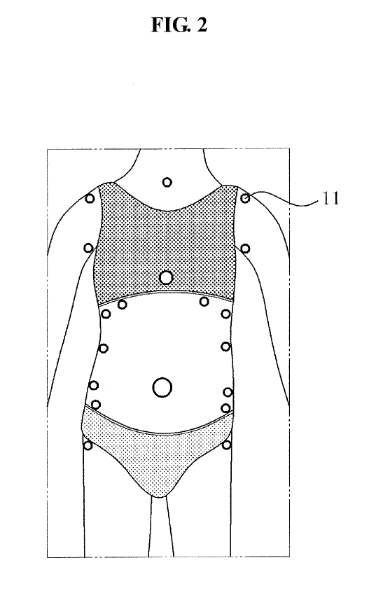

For example, as illustrated in FIG. 2, the plurality of markers 11, that is, at least one marker, is attached to the location of the skeleton based on the desired biochemical information. FIG. 2 is a diagram illustrating an example of attachment location of each marker to collect biochemical information.

The lower body trace unit 10 includes a plurality of markers 11 and image data collection unit 13. The plurality of markers 11 is attached to a lower body of the measurement target, and the image data collection unit 13 traces the location of each of the plurality of markers 11 according to the standard movement.

To obtain the lower body biochemical information, the plurality of markers 11 is attached to a location of a skeleton of any one of feet, legs, and waist considering desired biochemical information.

The lower body trace unit 10 further includes a pressure data measurement unit 15 to measure a foot pressure of the measurement target according to the standard movement. The pressure data measurement unit 15 uses a force plate measuring pressure transferred from a sole of the measurement target.

The image data collection unit 13 used in the upper body trace unit/lower body trace unit 10 traces the location of each of the plurality of markers 11 attached to the measurement target while the measurement target performs the standard movement. The image data collection unit 13 uses at least one of infrared Charge-Coupled Device (CCD) camera, infrared Complementary Metal Oxide Semiconductor (CMOS) camera, general CCD camera, and general CMOS camera depending on a type of the marker 11. Alternatively, the image data collection unit 13 uses a stereo camera using at least two cameras described above. For example, when an infrared marker is used as the plurality of markers 11, the image data collection unit 13 uses the infrared CCD/CMOS camera or stereo camera. When the color marker is used, the image data collection unit 13 uses the general CCD/CMOS camera or stereo camera. The biomechanical data and parameter analysis unit 30 includes a data/parameter calculation unit 31 and monitor 33. The data/parameter calculation unit 31 includes a data calculation unit and a biochemical parameter calculation unit. The data calculation unit associates the upper body biochemical information with lower body biochemical information, measured by the upper body trace unit/lower body trace unit 10, and calculates the associated biochemical information as 3D static data or 3D motion tracking data. The biochemical parameter calculation unit calculates biochemical parameters to be clinically applied using the 3D static data or 3D motion tracking data. The monitor 33 visualizes the 3D static data or 3D motion tracking data, and the biochemical parameter calculated by the data/parameter calculation unit 31. Also, the monitor 33 simultaneously displays the upper body biochemical information and lower body biochemical information. The data calculation unit and biochemical parameter calculation unit in the data/parameter calculation unit 31 may be divided and independently operated.

The data/parameter calculation unit 31 includes an upper body information modeling module, lower body information modeling module, biochemical data analysis module, and biochemical parameter analysis module. The upper body information modeling module collects the upper body biochemical information measured with respect to the standard movement and models the collected upper body biochemical information on a time axis. The lower body information modeling module collects the lower body biochemical information measured with respect to the standard movement and models the collected lower body biochemical information on the time axis. The biochemical data analysis module synchronizes the modeled upper body biomechanical information and lower body biomechanical information, combines the upper body biomechanical information and lower body biomechanical information, and outputs the 3D static data or 3D motion tracking data which is the combined information. The biochemical parameter analysis module outputs a biochemical parameter enabling a standard recommended prescription using the biochemical data.

According to an embodiment of the present invention, since the upper body biochemical information and lower body biochemical information is simultaneously provided, the upper body trace unit and lower body trace unit are required to be synchronized. However, it is apparent that synchronization is not required when the upper body trace unit and lower body trace unit are performed in a single computer system.

A method which sets a master clock from among the upper body trace unit and lower body trace unit is applied for the synchronization. A biochemical analysis unit may be the master clock.

Specifically, the upper body trace unit and lower body trace unit may be assigned as the master clock or slave, respectively, or a trace unit which is first operated from among the upper body trace unit and lower body trace unit may be set as the master clock on its own.

When any one of the upper body trace unit and lower body trace unit is set as the master clock, the clock master transmits a clock synchronization signal to the other trace unit to synchronize the upper body trace unit and lower body trace unit.

That is, the biochemical information obtained by the upper body trace unit and lower body trace unit is synchronized, and the upper body biomechanical information and lower body biomechanical information may be stored over time.

Other synchronization methods well-known in related arts besides the synchronization method described above may be applied.

The above-described 3D biochemical data and parameter analysis system is provided to experts including doctors (hereinafter, referred to as ‘expert’).

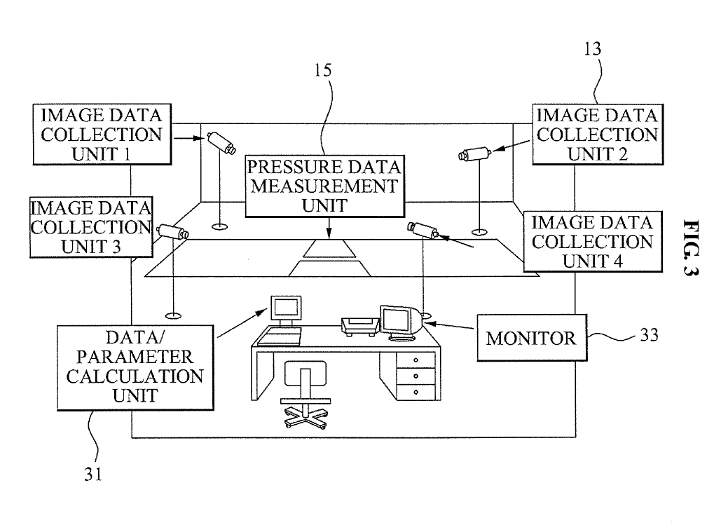

FIG. 3 is a diagram illustrating a placement of the 3D biochemical data and parameter analysis system of FIG. 1.

In the 3D biochemical data and parameter analysis system, each unit is placed in a predetermined location, and set to enable the 3D biochemical data and parameter analysis system to be performed.

As illustrated in FIG. 3, a pressure data measurement unit 15 is installed, and a plurality of image data collection units 13 is installed in various directions around the pressure data measurement unit 15. The pressure data measurement unit 15 measures foot pressure of a measurement target. Also, the plurality of image data collection units 13 may be installed to calculate data and parameters of a specific body part with a low cost.

A plurality of markers 11 is attached to a location of the measurement target based on desired biochemical information. The measurement target performs a standard movement on the pressure data measurement unit 15. When information about the foot pressure of the measurement target is not required, the pressure data measurement unit 15 may be deleted.

The plurality of image data collection units 13 measures a location of each of the markers 11 according to the standard movement. The pressure data measurement unit 15 measures the foot pressure of the measurement target according to the standard movement.

A 3D biomechanical data and parameter analysis unit 30 is provided to receive data, obtained from the plurality of image data collection units 13 and pressure data measurement unit 15, and provide the data to the expert. The 3D biomechanical data and parameter analysis unit 30 includes a data/parameter calculation unit 31 and monitor 33.

The data/parameter calculation unit 31 and monitor 33 may be separately installed or integrally configured in a single terminal.

The data of the plurality of image data collection units 13 and pressure data measurement unit 15 is transmitted to the data/parameter calculation unit 31. Also, an input/output of the data may or may not be synchronized.

Hereinafter, a 3D biochemical data and parameter analysis method is described in detail.

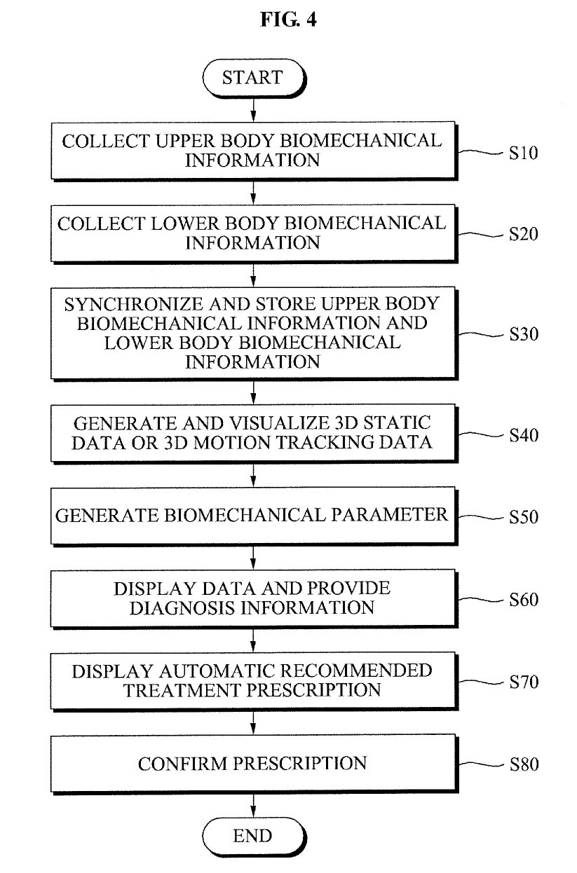

FIG. 4 is a flowchart illustrating a 3D biochemical data and parameter analysis method according to an embodiment of the present invention.

According to an embodiment of the present invention, upper body biochemical information and lower body biochemical information are simultaneously collected, associated with each other, and provided.

That is, it is preferable that at least one upper body biochemical information and lower body biochemical information about at least one part are simultaneously collected.

An expert attaches markers 11 to an upper body and lower body and directs a standard movement considering both the upper body and lower body in order to obtain biochemical information about the upper body and lower body through the markers 11. Specifically, the standard movement is displayed through a display unit such as a video, and thus the measurement target performs the standard movement. Also, the markers 11 are attached to the upper body and lower body, and the measurement target is directed to perform the standard movement on a pressure data measurement unit 15 while the markers 11 are attached to the measurement target in order to obtain the biochemical information including the foot pressure of the measurement target.

In operation S10, upper body biochemical information measured by an image data collection unit 13 according to the standard movement is collected.

In operation S20, lower body biochemical information measured by the image data collection unit 13 or/and pressure data measurement unit 15 according to the standard movement is collected.

In operation S30, the collected upper body biochemical information and lower body biochemical information are synchronized on a time axis and stored.

In operation S40, the stored upper body biochemical information and lower body biochemical information are associated with each other, and thus 3D static data or 3D motion tracking data are generated, visualized and stored.

In operation S50, a biochemical parameter to be clinically applied is generated using the generated 3D static data or 3D motion tracking data, and the data is stored.

In operation S60, the 3D static data or 3D motion tracking data, upper body biochemical information and lower body biochemical information, average value and distribution value of biochemical parameters of each part, and a value obtained while performing the standard movement are displayed through a monitor 33. Also, in operation S60, a result of a diagnosis and prognosis of a biochemical musculoskeletal system disease based on the biochemical parameter is provided. The 3D static data or 3D motion tracking data is compared with a normal reference data with respect to the standard movement, and thus diagnosis information, for example, leading prescription, spine orthosis prescription, rehabilitation, surgical operation, and the like, may be provided according to a result of the comparing.

In operation S70, an automatic recommended treatment prescription is displayed on the monitor 33 based on the biochemical parameter. Also, in operation S70, the method enables the expert to change the automatic recommended treatment prescription based on the automatic recommended treatment prescription, upper body biochemical information, lower body biochemical information, and biochemical parameter.

In operation S80, when the leading prescription and/or spine orthosis prescription is supplied, the upper body biochemical information and lower body biochemical information are collected again and it is confirmed whether the prescription is supplied as prescribed. When the prescription was not supplied as prescribed, a prescription is supplied again.

Accordingly, the expert may determine a comprehensive diagnosis and validity of prescription considering both the upper body biochemical information and lower body biochemical information three-dimensionally displayed on the monitor 33.

Hereinafter, a method of obtaining information about the biochemical parameters using the upper body biochemical information and lower body biochemical information is described in detail.

First, to obtain biochemical data for each biochemical parameter, it is preferable that a marker is attached to a skeleton and/or muscle area corresponding to an anatomical location of a measurement target. The skeleton and/or muscle area corresponds to the biochemical data.

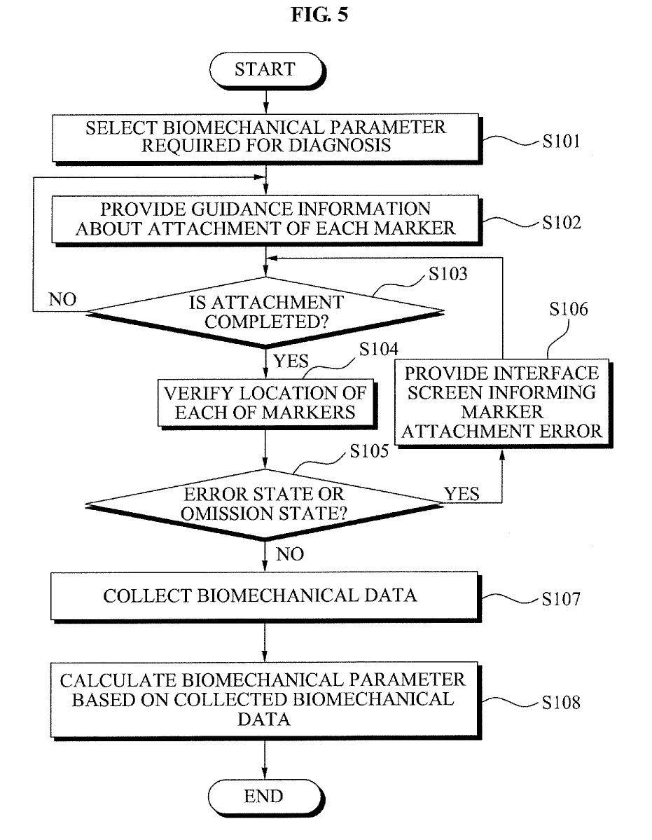

FIG. 5 is a flowchart illustrating a method of collecting biochemical data in a 3D biomechanical data and parameter analysis system using a marker according to an embodiment of the present invention.

In operation S101, at least one biomechanical parameter required to diagnose a musculoskeletal system disease of a measurement target is selected.

In this instance, an expert may execute an interface screen, and select the biochemical parameter through the interface screen.

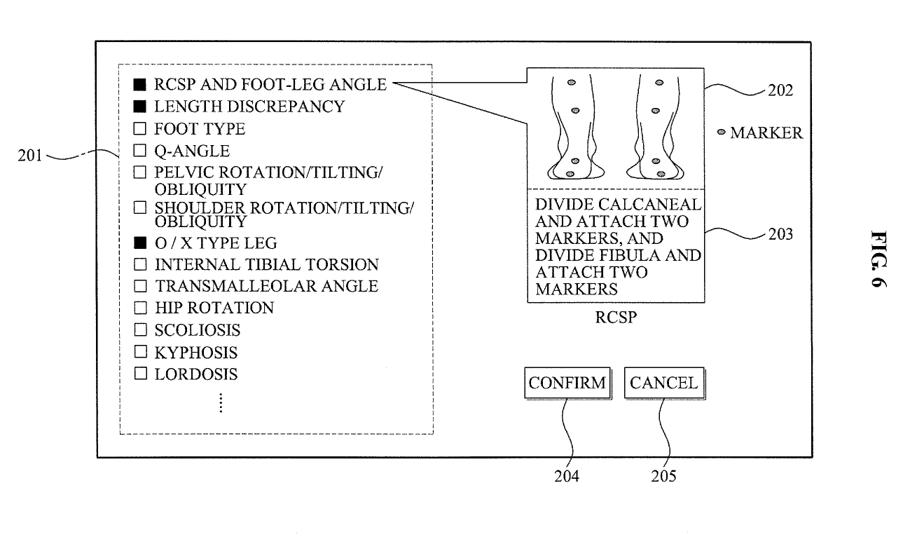

FIG. 6 is a diagram illustrating an example of an interface screen to provide an attachment location of each marker and biochemical parameter list 201.

As illustrated in FIG. 6, the interface screen may include the biochemical parameter list 201 providing a list of the biochemical parameters described above. The biochemical parameter list 201 may include a selection menu for each biochemical parameter to enable the expert to select at least one biochemical parameter.

The interface screen may further include a guidance menu. The guidance menu provides guidance about the attachment location of each of the markers, that is, biochemical data corresponding to the biochemical parameter selected by the expert.

The guidance menu may include a marker marked area 202 and a phrase mark area 203. The marker marked area 202 is for displaying a marker on an area of a predetermined human body shape by using the human body shape as a model. The area corresponds to the determined location of each of the markers. The phrase mark area 203 is for displaying a phrase describing the attachment location of each of the markers.

The interface screen may further include a confirm button 204 and cancel button 205. The confirm button 204 is for inputting a selection on the biochemical parameter after selecting the biochemical parameter from the biochemical parameter list 21. The cancel button 205 is for terminating an execution of the interface screen.

As described above, when the expert selects at least one biochemical parameter from the biochemical parameter list 201 and inputs the confirm button 204, an operation of selecting the biochemical parameter is completed.

Referring again to FIG. 5, in operation 5102, when the operation of selecting is completed, guidance information about an attachment of the markers is provided based on biochemical data corresponding to the biochemical parameter selected by the expert.

The guidance information about the attachment of the markers may include the attachment location of each of the markers and the phrase describing the location of each of the markers to collect the biochemical data.

The biochemical data is previously defined through a predetermined clinical test. Accordingly, it is preferable that a database is established. The database previously stores biochemical data for each biochemical parameter and the attachment location of each of the markers.

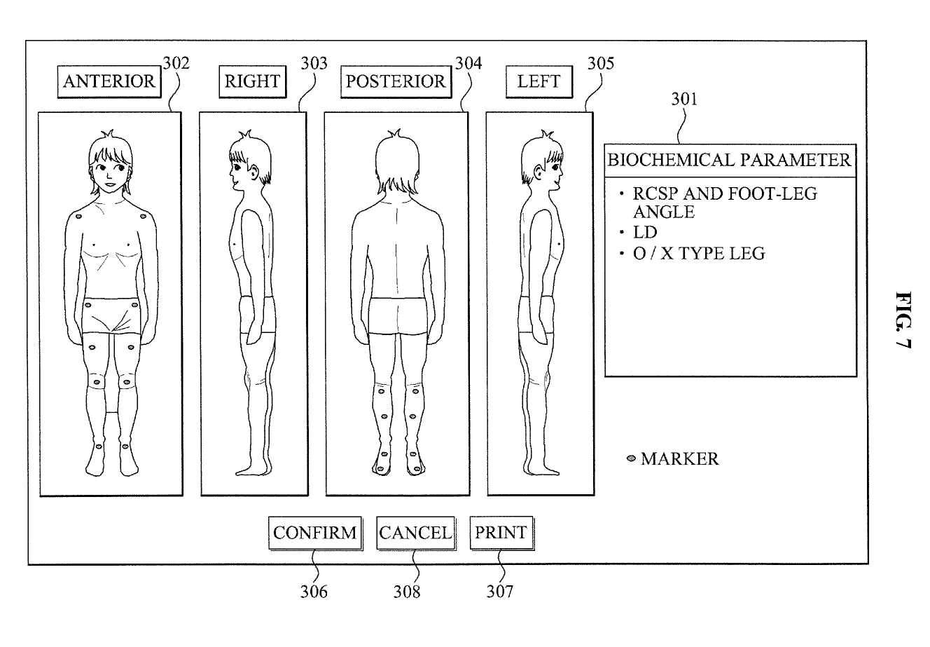

FIG. 7 is a diagram illustrating an example of an interface screen to provide an attachment location of each marker to be attached to an anterior, posterior, right, and left side of a measurement target.

When an expert inputs a confirm button 204 after selecting a biochemical parameter through an interface screen of FIG. 6, an interface screen illustrated in FIG. 7 is displayed.

The interface screen illustrated in FIG. 7 may provide guidance about an attachment location of each of the markers with respect to each side by dividing a human body shape into four sides, that is, anterior, posterior, left, and right side.

The interface screen may include a biochemical parameter list 301 selected by the expert, marker location of the anterior side 302, marker location of the right side 303, marker location of the posterior side 304, and marker location of the left side 305. The interface screen is configured based on the attachment location of each of the markers for each biochemical parameter stored in the database.

The interface screen may further include a confirm button 306 and print button 307. Also, the interface screen may further include a cancel button 308. The confirm button 306 is for inputting about completion of the attachment of markers. The print button 307 is for printing a hardcopy of the interface screen providing the attachment location of the markers. The cancel button 308 is for terminating an execution of the interface screen or moving backward to a previous screen.

The expert attaches the markers to each skeleton part of the measurement target by referring to the marker location of the anterior side 302, marker location of the right side 303, marker location of the posterior side 304, and marker location of the left side 305.

When the expert inputs a command with respect to termination of the attachment of the markers, for example, when the expert inputs the confirm button 306 after completing the attachment in operation S103, the location of each of the markers attached to the skeleton of the measurement target is verified in operation S104.

In the verifying in operation S104, the location of each of the markers attached to the skeleton of the measurement target is sensed using an image data collection unit, and the sensed location and a marker location provided through the interface screen are compared.

In the verifying in operation S104, the sensed location and a marker location provided through the interface screen are compared, and an omission state or error state is determined. The omission state indicates that the marker is not attached, and the error state indicates that the sensed location is outside a predetermined range.

In the verifying in operation S104, the measurement target is divided into the anterior, posterior, right, and left side, and the location of each of the markers may be sequentially verified with respect to each side. Alternatively, the location of each of the markers may be simultaneously verified with respect to two sides or every side.

The omission state or error state is sensed in operation S105 while verifying, the interface screen informing a marker attachment error is provided in operation S106.

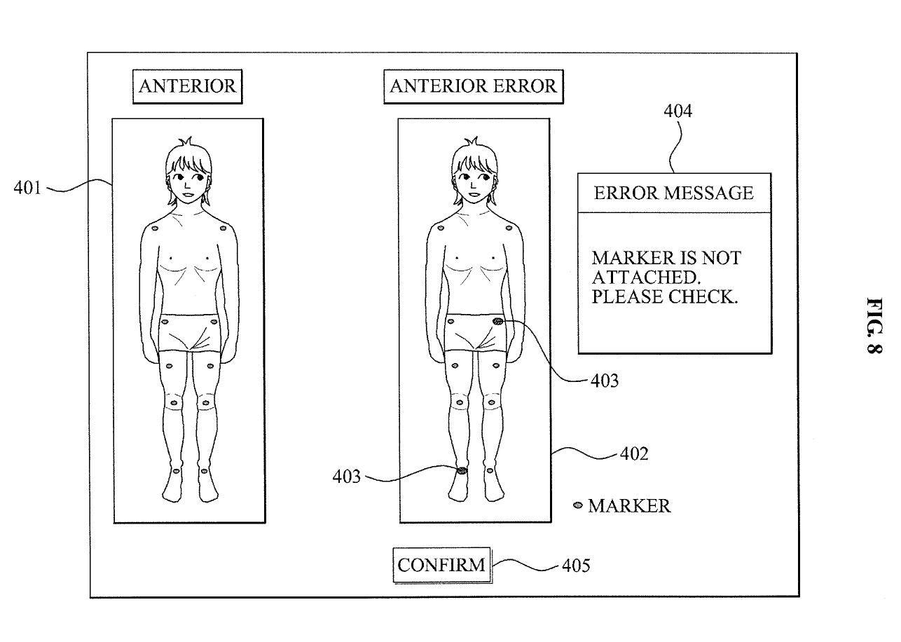

FIG. 8 is a diagram illustrating an example of an interface screen to provide an error state or omission state of a marker while verifying a location of each marker attached to a measurement target.

The interface screen may include a location verification mark area 401 and error location mark area 402. The location verification mark area 401 is for displaying a side where a location verification is performed from among an anterior, posterior, right, and left side of the measurement target. The error location mark area 402 is for informing about a marker 403 where the omission state or error state is sensed from among the markers of the side.

The interface screen may further include an error message mark area 404. The error message mark area 404 is for informing the omission state or error state of the marker 403 and displaying a variety of messages requesting an expert for reattachment.

The interface screen may further include a confirm button 405. The confirm button 405 is used to request the location verification again after the expert checks the marker 403 where the omission state or error state is sensed.

When the location verification is completed as described above, all the markers attached to a skeleton of the measurement target are sensed, and thus biochemical data is collected in operation S107. In operation S108, a biochemical parameter to diagnose a musculoskeletal system disease is calculated based on the biochemical data. Hereinafter, a method of extracting biochemical data of upper body biochemical information and lower body biochemical information is described in detail.

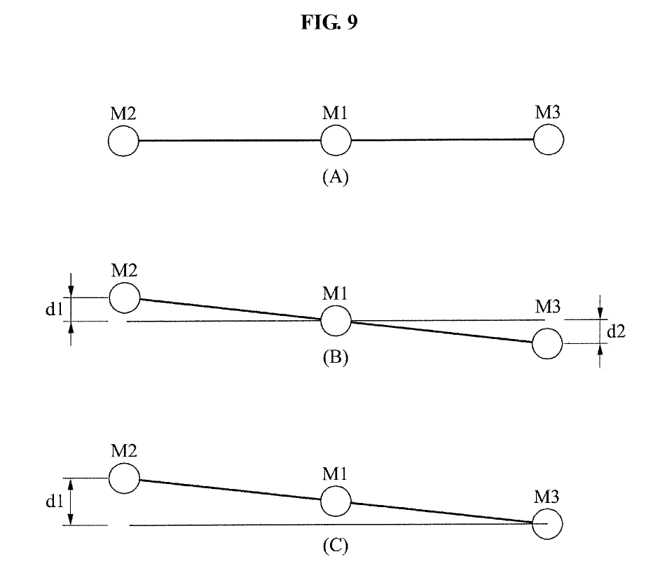

By referring to FIG. 9, a method of measuring a shoulder elevation included in upper body biochemical information is described.

Markers 11 are attached to outer points M2 and M3 of each scapula and a center position M1 of both shoulder blades.

A location information image of the markers 11 is used, and the location information image is received from an image data collection unit 13. As illustrated, a connection line connecting points M1, M2, and M3 of each of the markers 11 and reference data (horizon) are compared. Also, a difference in height between the reference data and the connection line is measured. Accordingly, the shoulder elevation may be calculated.

FIG. 9 (A) illustrates the connection line among the points M1, M2, and M3 is identical to the reference data. FIG. 9 (B) illustrates a height difference dl at the point M2 and a height difference d2 at the point M3 exist. FIG. 9 (C) illustrates a height difference dl at the point M2 exists.

The method of measuring the shoulder elevation includes a method using a number of pixels, and a method using two-dimensional (2D) or 3D coordinates based on meter unit.

First, the method using the number of pixels may easily calculate the difference in height between the outer points M2 and M3 of the shoulder blades using the number of pixels when the markers 11 at the points M1, M2, and M3 are in a same plane. When a distance for each pixel is a (mm), a distance of a number of pixels d1 is d1 * a (mm).

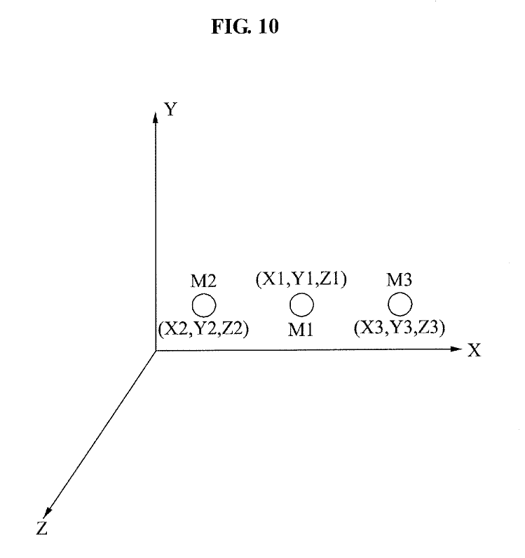

Also, the location of each of the points M1, M2, and M3 may be displayed as 2D coordinates or 3D coordinates. FIG. 10 illustrates an example of a location of each marker in 3D coordinates. When 3D coordinates of each of the points M1, M2, and M3 is (x1, y1, z1), (x2, y2, z2), and (x3, y3, z3), a difference in height of the outer points M2 and M3 of the shoulder blades may be calculated as □y3-y2□. Here, □□ indicates an absolute value.

It is apparent that mathematical methods besides the methods described above may be used, and thus the present invention is not limited to the described exemplary embodiments.

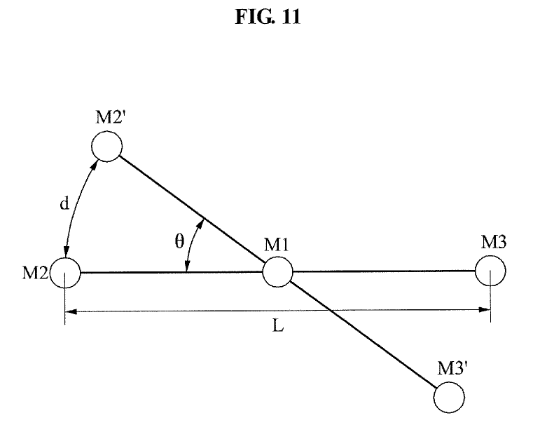

By referring to FIG. 11, a method of measuring degree of shoulder rotation of the biochemical information is described. Rotation generally indicates a degree of change in coordinates of an axis z. It is apparent that a value of an axis x and a value of an axis y may change.

Markers 11 are attached to outer points M2 and M3 of each shoulder blade and a center position M1 of both shoulder blades.

As illustrated, a movement of the markers 11 at the points M2 and M3 is observed based on location information of the markers 11 with respect to a standard movement. The location information is received from an image data collection unit 13. An angle θ among points M2′, M1, and M2 is calculated using movement amount d of the markers 11 at the points M2 and M3 with respect to the standard movement. The movement amount d indicates a distance measured from when a measurement target moves in an original direction to when the measurement target moves in opposite direction with respect to the standard movement. That is, when a point turning the opposite direction from the original direction is the point M2, a point turning the original direction from the opposite direction is M2′.

When the movement amount d is calculated using the number of pixels, an actual distance where the measurement target moves is d * a (mm). In this instance, a distance for each pixel is a (mm). Also, when a distance L between the point M2 and point M3 is calculated using the number of pixels, an actual distance is L * a (mm). Accordingly, the angle θ is d/L.

Also, the 3D coordinates may be used. Coordinates of each of the points M1, M2, and M3 is (x1, y1, z1), (x2, y2, z2), and (x3, y3, z3), respectively. Coordinates of the point M2′ is (x2′, y2′, z2′), and coordinates of a point M3′ is (x3′, y3′, z3′). To easily obtain an angle of the 3D coordinates, the point M2, point M2′, point M3, and point M3′ are displaced in parallel so that the point M1 is to be an origin O of the coordinate system. Accordingly, new coordinates of the point M1. is O (0, 0, 0), new coordinates of the point M2 is M2 (x2-x1, y2-y1, z2-z1), and new coordinates of the point M2′ is M2′ (x2′-x1, y2′-y1, z2′-z1). Thus, an angle between a line 0M2 and the axis x is tan⁻¹{(z2-z1)/(x2-x1)}, and an angle between a line 0M2′ and the axis x is tan⁻¹{(z2′-z1)/(x2′-x1)}. An angle among the points M2, O, and M2′ is [tan⁻¹{(z2-z1)/(x2-x1)}-tan⁻¹{(z2-z1)/(x2-x1)}]. Thus, the angle θ among the points M2′, M1, and M2 is the same as an angle among the points M2, O, and M2′, which is [tan⁻¹{(z2-z1)/(x2-x1)}-tan⁻¹{(z2-z1)/(x2-x1)}].

That is, to obtain the degree of shoulder rotation, location information image of the markers 11 is periodically collected, and the collected location information image is modeled on a time axis. The location information image of the markers 11 is received from the image data collection unit 13 until the standard movement finishes. The degree of shoulder rotation with respect to the standard movement is 3D motion tracking data.

A pelvis elevation and degree of pelvis rotation of the upper body biochemical information may be measured in a similar way to the method of measuring the shoulder elevation and shoulder rotation described above. Also, since calculating a tilting degree of the shoulder and pelvis is calculating an angle, the calculation of the tilting degree is similar to the method of measuring the rotation. When measuring the rotation, the point M1 is a reference point. However, when measuring the tilting degree of the pelvis, a reference point is a point where a pelvis and spine are joined. The reference point described above is simply an example, and the tilting degree of the shoulder and pelvis may be calculated using other reference points.

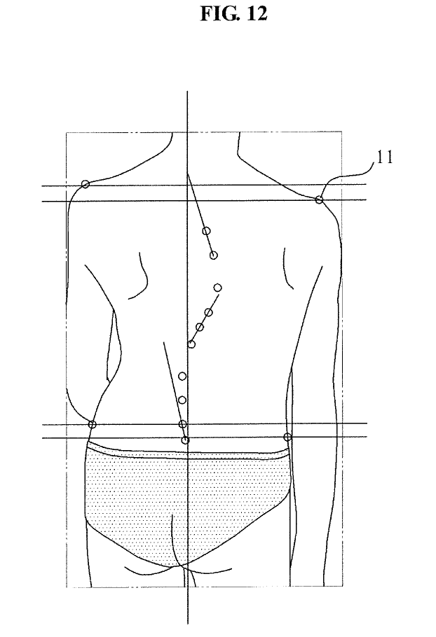

FIG. 12 illustrates an image where a location of each of a plurality of markers 11 attached to a spine is photographed.

To measure a curvature direction of the spine and degree of curvature of the spine, a method where the plurality of markers 11 is attached to a skeleton in the spine as illustrated in FIG. 12 and a location of each of the attached markers 11 is photographed is used. A method where a line connecting the location of each of the attached markers 11 and reference data (vertical line) are compared may be used to measure the curvature direction of the spine, degree of curvature of the spine, and the like.

The methods described above are easier and more accurate than an X-ray photograph image with respect to a diagnosis and prognosis of a biochemical musculoskeletal system disease.

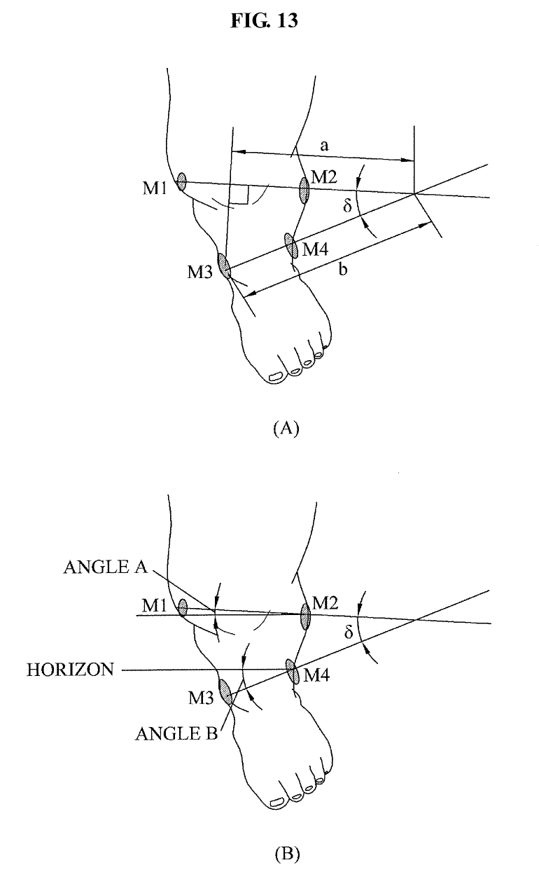

By referring to FIG. 13, a method of measuring an angle between an anklebone and knee joint of lower body biochemical information is described. To measure the angle between the anklebone and knee joint, markers are attached to both points M1 and M2 of the knee joint and both points M3 and M4 of the anklebone of a measurement target.

As illustrated, an angle S between a first extension line and a second extension line is calculated. The first extension line is generated by connecting the points M1 and M2 of the knee joint, and the second extension line is generated by connecting the points M3 and M4 of the anklebone. Accordingly, the angle between the anklebone and knee joint is calculated. For reference, when the first extension line and second extension line are projected to XZ plane coordinates, the angle S may be calculated. When the angle δ is on a right side, the angle δ has a positive value, and when the angle δ is on a left side, the angle δ has a negative value. In general, the first extension line is referred to as a Transcoudylar line, and the second extension line is referred to as a Transmalleolar line.

As illustrated in FIG. 13 (A), a length a and length b are calculated using an image showing the markers 11 at the points M1, M2, M3, and M4, and then the angle δ may be calculated. Also, as illustrated in FIG. 13 (B), the angle δ may be calculated using an equation, that is, angle δ=angle A+angle B. The angle A is between the first extension line and reference data (horizon), and the angle B is between the second extension line and the reference data (horizon). The angle A and angle B are obtained as follows: The points M1, M2, M3, and M4 are used. A slope between two lines, that is, a line between the point M1 and point M2 and a line between the point M3 and point M4, may be calculated using coordinates of each of the two lines. Accordingly, the angle A and angle B may be obtained. In particular, a method of measuring severity of Internal Tibial Torsion is used to calculate the angle 5. When the coordinates is calculated using a contact or non-contact method including vision, the angle δ may be obtained through the above-described method.

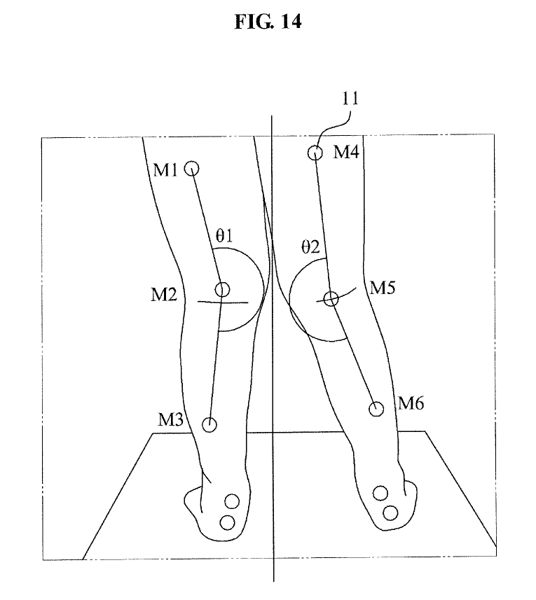

By referring to FIG. 14, a method of measuring an angle between a femur and a fibula of lower body biochemical information is described in detail.

As illustrated, markers 11 are attached to upper parts M1 and M4 of each femur, a center of each knee joint M2 and M5, and lower parts M3 and M6 of each fibula.

An angle θ1 and θ2 between the femur and fibula of each leg is calculated using an image showing a location of each of the markers 11. The image is received from an image data collection unit 13. As illustrated in FIG. 14, a first extension line and second extension line are used. The first extension line is generated by connecting the points M1, M2, and M3, and the second extension line is generated by connecting the points M4, M5, and M6. An angle θ1 between the first extension line and reference data, for example, a vertical line, and angle θ2 between the second extension line and the reference data are calculated. Accordingly, the angle between the femur and fibula may be calculated.

Hereinafter, a method of measuring pelvis-related biochemical data of lower body biochemical information is described in detail.

The pelvis-related biochemical data may include a pelvis rotation angle, pelvis obliquity angle, pelvis tilting angle, and pelvis elevation.

The pelvis-related biochemical data may be calculated using static data of a predetermined biochemical measurement point. The static data may be set as an initial value of the pelvis-related biochemical data.

Specifically, a left/right Posterior Superior Iliac Spine (PSIS), Anterior Superior Iliac Spine (ASIS), and left/right Iliac Crest (IC) of a measurement target are required to be measured in a static state to calculate the pelvis-related biochemical data. Since the left/right PSIS and the left/right ASIS are in different locations, the left/right PSIS and the left/right ASIS may not be measured using a single image data collection unit. Accordingly, after the left/right PSIS, the left/right ASIS and the left/right IC are measured using at least two image data collection units, locations of the left/right PSIS and the left/right ASIS are measured using the left/right IC. When using the single image data collection unit, the left/right PSIS and the left/right IC, and the left/right

ASIS and the left/right IC are measured, respectively. Accordingly, a relationship between the left/right PSIS and the left/right ASIS may be ascertained using the left/right PSIS, the left/right ASIS, and the left/right IC measured in the static state.

Since left/right pelvis actually consists of a single bone, a location relationship among IC, PSIS, and ASIS does not change. That is, when a location of each of the PSIS, IC, and ASIS is measured, the relationship between the PSIS and ASIS may be ascertained using the IC.

To ascertain the relationship between the PSIS and ASIS, a relationship (X, Y, Z) between the ASIS and a Front IC (Left, Right) is calculated through a photographed image of an anterior side of the measurement target, and a relationship (X′, Y′, Z′) between the PSIS and a Back IC (Left, Right) is calculated through a photographed image of a posterior side of the measurement target. In this instance, the relationship between the PSIS and ASIS may be ascertained by matching location coordinates of the Front IC and Back IC.

For example, when location coordinates of the Front left IC is (−10, 10, 10), location coordinates of the ASIS is (0, 0, 0), location coordinates of the Back left IC is (−20, 20, 20), and location coordinates of the PSIS is (0, 0, 0), the Front left IC and Back left IC are to be on a same line by moving the location coordinates of the Front left IC to (−10, 10, 10) in order to ascertain a relative location of the ASIS based on the PSIS. In this instance, since the ASIS moves as much as the Front left IC moves, the ASIS moves to (−10, 10, 10) from (0, 0, 0). Also, the ASIS is located on the location coordinates (−10, 10, 10) based on the PSIS.

Also, when the static data is used as the initial value, pelvis-related biochemical data with respect to a dynamic state of the measurement target may be obtained. That is, when the left/right PSIS and left/right IC in the dynamic state are obtained, a location of the ASIS may be estimated using the location relationship between the PSIS and ASIS measured in a static state. Similarly, when the left/right ASIS and the left/right IC are measured, a location of the left/right PSIS may be ascertained using the location relationship between the PSIS and ASIS.

Hereinafter, a method of measuring pelvis-related biochemical data in the dynamic state is described in detail.

A location change of the PSIS (or ASIS) and IC in the dynamic state is required to be measured to calculate the pelvis-related biochemical data in the dynamic state. Measurement points of the measurement target are a left/right PSIS or left/right ASIS, center position of PSIS or ASIS, and left/right IC. In this instance, the center position of the PSIS or ASIS may be measured by directly measuring and measuring the center position using the measured left/right PSIS or ASIS.

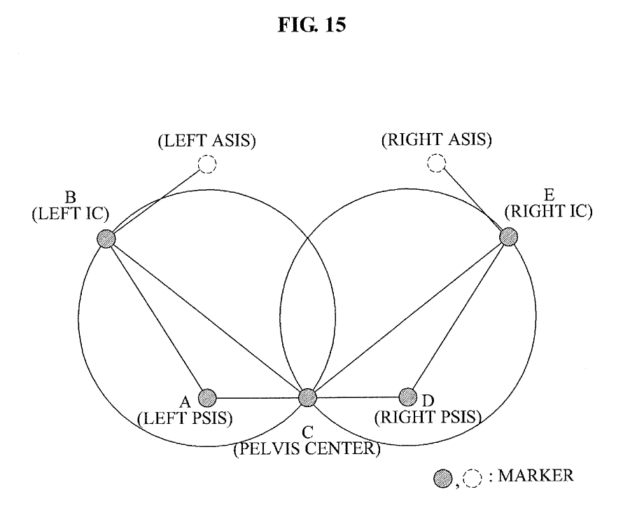

FIG. 15 is a diagram illustrating a measurement point of pelvis-related measurement point.

As illustrated, a left PSIS A, right PSIS D, left IC B, and right IC E may be measured in a posterior side of a measurement target, and right/left ASIS, left IC B, and right IC E may be measured in an anterior side of the measurement target. The left IC B and right IC E are symmetrically in a same position. Also, the left PSIS, left IC, and left ASIS, and the right PSIS, right IC, and right ASIS are on a single bone. Accordingly, a relationship between the ASIS and IC may be measured using a relationship between the PSIS and IC. Also, since location information about a left biochemical measurement point and right biochemical measurement point may be different, it is preferable that pelvis-related biochemical data of a left side and pelvis-related biochemical data of a right side are separately calculated. A method of calculating pelvis-related biochemical data of the left side is identical to a method of calculating pelvis-related biochemical data of the right side.

In an embodiment of the present invention, a method of calculating the pelvis-related biochemical data of the left side using a location change of the left PSIS and left IC is described.

First, location information about the left PSIS A, left IC B, and center position C of the PSIS with respect to a standard movement in the static state is collected. The location information about the left PSIS A, left IC B, and center position C of the PSIS with respect to the standard movement in the static state is set as a reference point.

Also, to measure a periodical change in a dynamic state, location information about the left PSIS A, left IC B, and center position C of the PSIS is collected at predetermined intervals while the measurement target performs the standard movement in the dynamic state. Hereinafter, the location information is referred to as a moving point.

The reference point and moving point of the biochemical measurement point are mapped to a 3D coordinate system. Specifically, the location information about the left PSIS A, left IC B, and center position C of the PSIS, that is, the biochemical measurement point indicating the reference point or moving point, is mapped to the 3D coordinate system including an axis x (horizontal), axis y (height), and axis z (vertical). Here, the axis x indicates a left and right of a pelvis of the measurement target in an anterior side, the axis y indicates an upper and lower part of the pelvis, and the axis z indicates an anterior and posterior side of the pelvis.

The pelvis-related biochemical data may be calculated by measuring location change of the moving point with respect to the reference point of the biochemical measurement point using the 3D coordinate system. That is, the location change between the moving point and reference point with respect to a reference axis of the 3D coordinate system is measured, and thus the pelvis-related biochemical data such as a pelvis rotation angle, pelvis obliquity angle, pelvis tilting angle, and pelvis elevation may be calculated.

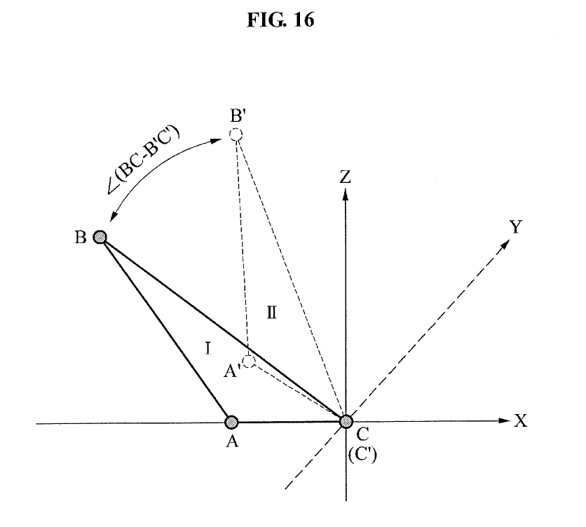

By referring to FIG. 16, a method of measuring calculating a pelvis rotation angle is described in detail.

To calculate the pelvis rotation angle, a first plane I and second plane II are mapped on a 3D coordinate system. The first plane I is generated by connecting a left PSIS A, left IC B, and center position C of PSIS, and the second plane If is generated by connecting a left PSIS A′, left IC B′, and center position C′ of the PSIS. The left PSIS A, left IC B, and center position C of the PSIS correspond to a reference point, and the left PSIS A′, left IC B′, and center position C′ of the PSIS correspond to a moving point.

Here, the center position C of the PSIS of the first plane I is matched with the center position C′ of the PSIS of the second plane II.

A location of the pelvis rotation angle does not change with respect to an axis y (height), and thus the pelvis rotation angle may be calculated by measuring an angle generated between the first plane I and second plane II with respect to the axis x (horizontal) and axis y (vertical) of the 3D coordinate system, that is, a location change between the reference point and moving point.

Specifically, an angle (∠(BC-B′C′)) between a line BC of the first plane I and a line B′C′ of the second plane II with respect to the axis x (horizontal) and axis z (vertical) is calculated. Accordingly, the calculated angle (∠(BC-B′C′)) is recognized as the pelvis rotation angle of the measurement target.

Or, an angle generated when barycentric coordinates of the first plane I moves to barycentric coordinates of the second plane II in an axis z direction based on coordinates of C(C′) is calculated. The calculated angle may be recognized as the pelvis rotation angle of the measurement target.

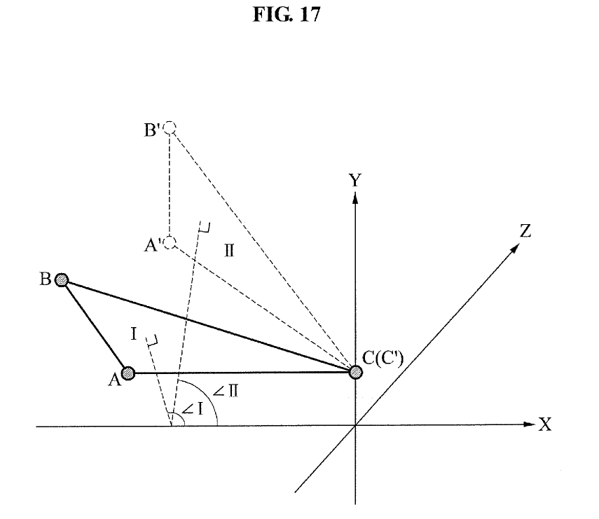

By referring to FIG. 17, a method of calculating a pelvis obliquity angle is described.

To calculate the pelvis obliquity angle, a first plane I and second plane II are mapped on a 3D coordinate system. The first plane I is generated by connecting a left PSIS A, left IC B, and center position C of PSIS, and the second plane II is generated by connecting a left PSIS A′, left IC B′, and center position C′ of the PSIS. The left PSIS A, left IC B, and center position C of the PSIS correspond to a reference point, and the left PSIS A′, left IC B′, and center position C′ of the PSIS correspond to a moving point.

Here, the center position C of the PSIS of the first plane I is matched with the center position C′ of the PSIS of the second plane II.

The pelvis obliquity angle may be calculated by measuring an angle generated between the first plane I and second plane II with respect to an axis x (horizontal) and axis y (height) of the 3D coordinate system, that is, a location change between a reference point and moving point.

Specifically, a first angle (∠I) and a second angle (∠II) are calculated. The first angle (∠I) is an angle between the axis x (horizontal) and a perpendicular line of the first plane I, and the second angle (∠II) is an angle between the axis x (horizontal) and a perpendicular line of the second plane II based on the axis x (horizontal) on the 3D coordinate system. A difference (∠I-∠II) between the calculated first angle (∠I) and second angle (∠II) is recognized as the pelvis obliquity angle of a measurement target. The perpendicular line of each of the first plane I and second plane II indicates a straight line perpendicular to a center of each of the first plane I and second plane II.

Alternatively, an angle (∠(BC-B′C′)) between a line BC of the first plane I and a line B′C′ of the second plane II with respect to the axis x (horizontal) and axis y (height) is calculated. Accordingly, the calculated angle (∠(BC-B′C′)) may be recognized as the pelvis obliquity angle of the measurement target.

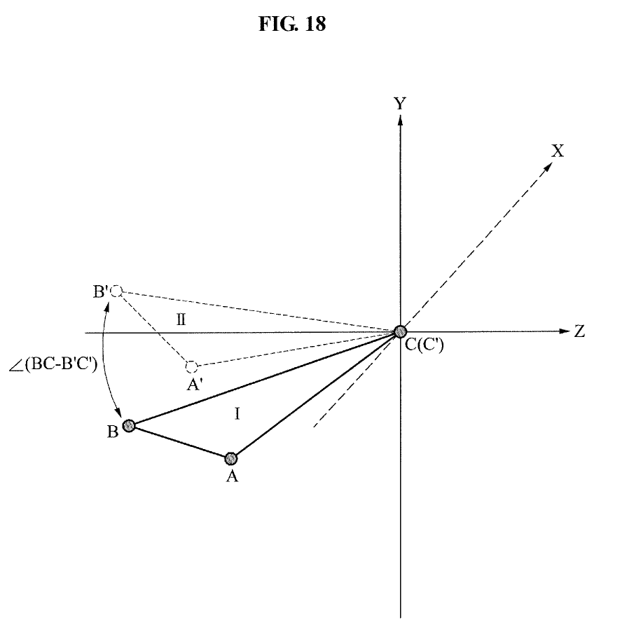

By referring to FIG. 18, a method of calculating a pelvis tilting angle is described.

To calculate the pelvis tilting angle, a first plane I and second plane II are mapped on a 3D coordinate system. The first plane I is generated by connecting a left PSIS A, left IC B, and center position C of PSIS, and the second plane II is generated by connecting a left PSIS A′, left IC B′, and center position C′ of the PSIS. The left PSIS A, left IC B, and center position C of the PSIS correspond to a reference point, and the left PSIS A′, left IC B′, and center position C′ of the PSIS correspond to a moving point.

Here, the center position C of the PSIS of the first plane I is matched with the center position C′ of the PSIS of the second plane II.

A location of the pelvis tilting angle does not change with respect to the axis x (horizontal), and thus the pelvis tilting angle may be calculated by measuring an angle generated between the first plane I and second plane II with respect to the axis z (vertical) and axis y (height) of the 3D coordinate system.

Specifically, an angle (∠(BC-B′C′)) between a line BC of the first plane I and a line B′C′ of the second plane II with respect to the axis z (vertical) and axis y (height) is calculated. Accordingly, the calculated angle (∠(BC-B′C′)) may be recognized as the pelvis tilting angle of the measurement target.

Or, a first angle (∠I) and a second angle (∠II) are calculated. The first angle (∠I) is an angle between the axis y (height) and a perpendicular line of the first plane I, and the second angle (∠II) is an angle between the axis y (height) and a perpendicular line of the second plane II based on the axis y (height) on the 3D coordinate system. A difference (∠I-∠II) between the calculated first angle (∠I) and second angle (∠II) is recognized as the pelvis tilting angle of the measurement target. The perpendicular line of each of the first plane I and second plane II indicates a straight line perpendicular to a center of each of the first plane I and second plane II.

The location change of a biochemical measurement point is periodically measured until a measurement target finishes a standard movement in a dynamic state, the location change is modeled on a time axis, and thus 3D motion tracking data such as a pelvis rotation angle, pelvis obliquity angle, and pelvis tilting angle with respect to the standard movement in the dynamic state may be calculated.

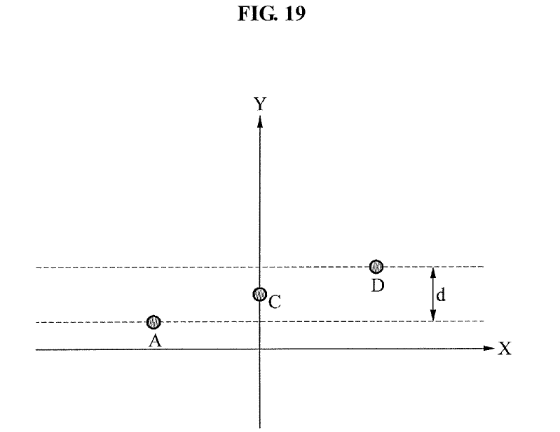

By referring to FIG. 19, a method of calculating a pelvis elevation is described.

To calculate the pelvis elevation, a left PSIS A, right PSIS D, and center position C of the PSIS is mapped on a 3D coordinate system. The left PSIS A, right PSIS D, and center position C of the PSIS correspond to a reference point.

Here, the center position C of the PSIS is matched with an origin of an axis y (height).

A coordinate value of the axis y of the left PSIS A and a coordinate value of the axis y of the right PSIS D are compared on the 3D coordinate system, and a difference value d between the coordinate values is measured to calculate the pelvis elevation.

According to an embodiment of the present invention, upper body biochemical information and lower body biochemical information are measured, respectively, the measured upper body biochemical information and lower body biochemical information are modeled on a time axis, and the upper body biochemical information and lower body biochemical information are synchronized. Also, a biochemical parameter to be clinically applied may be calculated using the biochemical information, and an automatic recommended treatment prescription is provided using the biochemical parameter.

An example of a diagnosis using the biochemical parameter and recommended treatment prescription is described.

Hereinafter, moving clockwise is considered as positive and moving counterclockwise is considered as negative with respect to a degree of rotation. A tilting degree when a measurement target bends forwards is positive and a tilting degree when the measurement target bends backwards is negative. It is negative that a left shoulder or left pelvis is higher than a right shoulder or pelvis, and it is positive that the left shoulder or left pelvis is lower than the right shoulder or pelvis with respect to a shoulder elevation and pelvis elevation. A direction is determined based on a posterior side of the measurement target.

Also, a type of feet is divided into an overpronation type, oversupination type, and normal pronation type. When the type is normal pronation type, the type of feet is divided into (pro, nor), (pro, sup), (nor, sup), (nor, pro), (sup, nor), and (sup, pro). In this instance, ‘pro’ indicates a pronation type, ‘sup’ indicates a supination type, and ‘nor’ indicates a normal type.

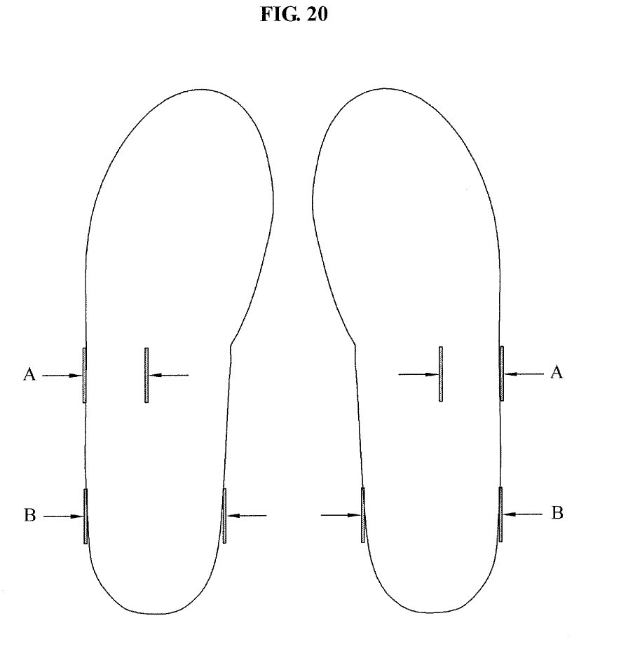

A method of determining the type of feet may be various. By referring to FIG. 20, a method of determining an overpronation type, oversupination type, and normal pronatino type is described.

FIG. 20 illustrates a foot pressure distribution using a foot pressure analyzer. In FIG. 20, a width A is a width of a narrowest part of a middle foot, and a width B is a width of a largest part of a rear foot.

That is, a length of a rear foot and middle foot is measured to divide a foot type.

In this instance, Arch Ration Index (ARI) is used, which is represented as,

ARI=(A/B)*100(%)

When the ARI is 0.6˜1, the foot type is the normal type, when the ARI is greater than 1, the foot type is the overpronation type, and when the ARI is less than 0.6, the foot type is the oversupination type.

Also, when the foot type is the normal pronation type, a relaxed calcaneal stance position (RCSP) angle is measured, and thus the pronation type, normal type, and supination type may be divided.

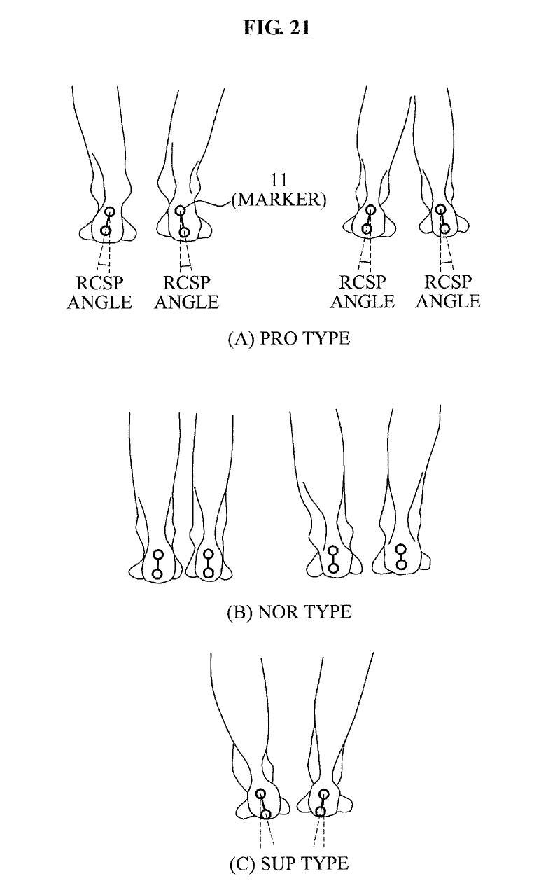

FIG. 21 illustrates a foot type, that is, a pronation type (A), normal type (B), and supination type (C).

When an RCSP angle is 0±1 degree (in some cases, 2 degrees is possible), the foot type may be the normal type. When the RCSP angle is greater than −1 degree (or −2 degrees), the foot type may be the pronation type. When the RCSP angle is +1 degree (or 2 degree), the foot type may be the supination type.

An operation of a recommended treatment prescription according to the above-described reference is described.

In the operation, it is ascertained whether a shoulder and pelvis elevation is positive or negative, shoulder and pelvis rotation degree is positive or negative, and shoulder and pelvis tilting degree is positive or negative.

Also, the foot type is determined from among the overpronation type, oversupination type, and normal pronation type. When the foot type is the normal pronation type, the feet type is determined from among (pro, nor), (pro, sup), (nor, sup), (nor, pro), (sup, nor), and (sup, pro).

Also, a number of results is generated through the operation, and an example of prescription on a few of the results is described.

For example, as a diagnosis result using a biochemical parameter, the pelvis elevation is positive, a value of the pelvis elevation is 6 mm, rotation degree of a right pelvis is −40 degrees (counterclockwise), rotation degree of a left pelvis is +20 degrees (clockwise), an RCSP angle of a right foot is −5 degrees, an RCSP angle of a left foot is 0 degree, and a foot type is the oversupination type, a leading prescription may be as follows: medial heel angle of 10 degrees, heel lift size 3 mm, inverted angle of 15 degrees, right posting angle varus of 4 degrees, and left posting angle of 0 degrees.

As another example, as a diagnosis result using the biochemical parameter, the pelvis elevation is negative, a value of the pelvis elevation is 1 cm, rotation degree of a left pelvis is 40 degrees, rotation degree of a right pelvis is −20 degrees, an RCSP angle of a left foot is −6 degrees, an RCSP angle of a right foot is +4 degrees, a foot type is the normal pronation type, and foot type of left foot and right foot is (pro, sup), a leading prescription may be as follows: medial heel angle of 4 degrees, heel lift size of 4 mm, inverted angle of 0 degrees, right posting angle valgus of 4 degrees, and left posting angle varus of 4 degrees.

According to the present invention, a 3D biochemical analysis system may diagnose a musculoskeletal system disease of a measurement target and transmit leading prescription data based on the diagnosis to a predetermined leading manufacture system, and thereby may automatically manufacture a leading of the measurement target.

The above-described embodiment of the present invention may be recorded in computer-readable media including program instructions to implement various operations embodied by a computer. The media may also include, alone or in combination with the program instructions, data files, data structures, and the like. The media and program instructions may be those specially designed and constructed for the purposes of the present invention, or they may be of the kind well-known and available to those having skill in the computer software arts. Examples of computer-readable media include magnetic media such as hard disks, floppy disks, and magnetic tape; optical media such as CD ROM disks and DVD; magneto-optical media such as floptical disks; and hardware devices that are specially configured to store and perform program instructions, such as read-only memory (ROM), random access memory (RAM), flash memory, and the like. Examples of program instructions include both machine code, such as produced by a compiler, and files containing higher level code that may be executed by the computer using an interpreter. The described hardware devices may be configured to act as one or more software modules in order to perform the operations of the above-described embodiments of the present invention.

Although a few embodiments of the present invention have been shown and described, the present invention is not limited to the described embodiments. Instead, it would be appreciated by those skilled in the art that changes may be made to these embodiments without departing from the principles and spirit of the invention, the scope of which is defined by the claims and their equivalents.

1. A three dimensional (3D) biomechanical data and parameter analysis system, comprising:
a biomechanical data and parameter analysis unit which collects first biomechanical information measured in an upper body and second biomechanical information measured in a lower body with respect to a standard movement, and provides 3D static data or 3D motion tracking data for a diagnosis or prognosis of a musculoskeletal system disease considering both the upper body and lower body, the 3D static data or 3D motion tracking data being generated by associating the first biomechanical information with second biomechanical information.

2. The 3D biomechanical data and parameter analysis system of claim 1, wherein the biomechanical data and parameter analysis unit comprises:
an upper body information modeling module which collects the first biomechanical information and models the collected first biomechanical information on a time axis while a measurement target performs the standard movement;
a lower body information modeling module which collects the second biomechanical information and models the collected second biomechanical information on the time axis while the measurement target performs the standard movement; and
a biomechanical analysis module which synchronizes the modeled first biomechanical information and second biomechanical information, combines the first biomechanical information and second biomechanical information, and outputs the 3D static data or 3D motion tracking data which is the combined information.

3. The 3D biomechanical data and parameter analysis system of claim 2, wherein the biomechanical data and parameter analysis unit further comprises a display module which visualizes the outputted 3D static data or 3D motion tracking data, and simultaneously provides the first biomechanical information and second biomechanical information.

4. The 3D biomechanical data and parameter analysis system of claim 1, further comprising:
an upper body trace unit which measures the first biomechanical information; and
a lower body trace unit which measures the second biomechanical information.

5. The 3D biomechanical data and parameter analysis system of claim 4, wherein the upper body trace unit comprises:
at least one marker which is attached to the upper body of the measurement target; and
a sensing unit which senses a location of the at least one marker according to the standard movement,
wherein the lower body trace unit comprises:
at least one marker which is attached to the lower body of the measurement target;
a sensing unit which senses a location of the at least one marker according to the standard movement; and
a pressure data measurement unit which measures foot pressure of the measurement target according to the standard movement.

6-13. (canceled)

14. The 3D biomechanical data and parameter analysis system of claim 2, wherein the biomechanical data and parameter analysis unit further comprises a diagnosis/prescription device which converts the first biomechanical information and second biomechanical information into a biochemical parameter to be clinically applied, and provides the diagnosis of the musculoskeletal system disease and a recommended prescription with respect to the diagnosed disease based on the biochemical parameter.

15. The 3D biomechanical data and parameter analysis system of claim 14, wherein the biomechanical data and parameter analysis unit further comprises a diagnosis/prescription device which re-collects and evaluates first biomechanical information and second biomechanical information which changes according to the recommended prescription, adjusts the recommended prescription, and provides the adjusted prescription.

16. A 3D biochemical data and parameter analysis method, comprising:
collecting first biomechanical information of an upper body and second biomechanical information of a lower body with respect to a standard movement; and
providing 3D static data or 3D motion tracking data for a diagnosis or prognosis of a musculoskeletal system disease considering both the upper body and lower body, the 3D static data or 3D motion tracking data being generated by associating the collected first biomechanical information with second biomechanical information.

17. The 3D biochemical data and parameter analysis method of claim 16, wherein the providing comprises:
modeling the collected first biomechanical information on a time axis;
modeling the collected second biomechanical information on the time axis;
synchronizing the modeled first biomechanical information and second biomechanical information to generate the 3D static data or 3D motion tracking data mutually associated with each other
visualizing the generated 3D static data or 3D motion tracking data, and simultaneously displaying the first biomechanical information and second biomechanical information; and
diagnosing the musculoskeletal system disease and determining a result of the prognosis based on the displayed first biomechanical information and second biomechanical information.

18-19. (canceled)

20. The 3D biochemical data and parameter analysis method of claim 16, wherein the collecting comprises:
setting an upper body trace unit and lower body trace unit to observe the upper body and lower body according to the standard movement;
measuring the first biomechanical information and second biomechanical information through the installed upper body trace unit and lower body trace unit; and
storing the first biomechanical information and second biomechanical information measured by the upper body trace unit and lower body trace unit.

21. The 3D biochemical data and parameter analysis method of claim 20, wherein the collecting further comprises:
determining any one of the upper body trace unit and lower body trace unit as a master clock for time synchronization;
providing, by the trace unit determined as the master clock, a clock synchronization signal to the other trace unit; and
synchronizing with the master clock according to the clock synchronization signal, dividing and storing the first biomechanical information and second biomechanical information chronologically.

22-24. (canceled)

25. The 3D biochemical data and parameter analysis method of claim 22, wherein the first biomechanical information is at least one of a degree of finger bending, degree of arm bone curvature, shoulder elevation, degree of shoulder tilting, curvature direction of a spine, and degree of curvature of the spin
wherein the second biomechanical information is at least one of a pelvis rotation angle, pelvis obliquity angle, pelvis tilting angle, pelvis elevation, degree of bending of lower limb, angle between a femur and a fibula, angle between a foot sole and vertex of calcaneal, angle between anklebone and knee joint, degree of toe bending, and foot pressure distribution.

26-27. (canceled)

28. The 3D biochemical data and parameter analysis method of claim 16, further comprising:
converting the first biomechanical information and second biomechanical information into a biochemical parameter to be clinically applied, and providing the diagnosis of the musculoskeletal system disease and a recommended prescription with respect to the diagnosed disease based on the biochemical parameter.

29. (canceled)

30. A 3D biochemical data and parameter analysis method, comprising:
selecting at least one biochemical parameter to be measured; and
providing guidance information about an attachment of markers to collect biochemical data corresponding to the selected biochemical parameter.

31. The 3D biochemical data and parameter analysis method of claim 30, wherein the selecting comprises:
providing an interface screen according to a command of a measurer; and
selecting at least one biochemical parameter required to diagnose a musculoskeletal system disease of a measurement target via the interface screen.

32. (canceled)

33. The 3D biochemical data and parameter analysis method of claim 31, wherein the interface screen further comprises a guidance menu which shows an attachment location of each of the markers based on the at least one selected biochemical parameter,
wherein the providing of the guidance information comprises:
determining the attachment location of each of the markers for each biochemical parameter selected from a list including the biochemical parameters; and
providing guidance about the determined attachment location of each of the markers via the guidance menu.

34-37. (canceled)

38. A 3D biochemical data and parameter analysis method, comprising:
determining a reference point with respect to a biochemical measurement point of a pelvis;
measuring a moving point of the biochemical measurement point due to a standard movement of a dynamic state; and
calculating biochemical data associated with the pelvis based on a location change between the moving point and reference point of the biochemical measurement point.

39. (canceled)

40. The 3D biochemical data and parameter analysis method of claim 38, wherein the biochemical data associated with the pelvis includes at least one of a pelvis rotation angle, pelvis obliquity angle, pelvis tilting angle, and pelvis elevation,
wherein the biochemical measurement point corresponds to a left or right Posterior Superior Iliac Spine (PSIS) or left or right Anterior Superior Iliac Spine (ASIS), center position of the left PSIS and right PSIS or center position of the left ASIS and right ASIS, and left or right Iliac Crest (IC), and
the determining comprises:
providing guidance about a standard movement of a static state to a measurement target;
measuring each point corresponding to the left or right PSIS or ASIS, center position of the left PSIS and right PSIS or center position of the left ASIS and right ASIS, and left or right IC due to the standard movement of the static state; and
determining the measured point as the reference point of the biochemical measurement point.

41. The 3D biochemical data and parameter analysis method of claim 38, wherein the measuring of the moving point comprises:
indicating the standard movement of the static state to a measurement target; and
measuring the moving point of the biochemical measurement point at predetermined intervals while the measurement target performs the standard movement of the dynamic state.

42. The 3D biochemical data and parameter analysis method of claim 40, wherein the calculating comprises:
mapping the moving point and reference point of the biochemical measurement point to a 3D coordinate system; and
calculating the biochemical data associated with the pelvis using the location change between the moving point and reference point of the biochemical measurement point.

43-48. (canceled)
